(19) United States
(12) Patent Application Publication
(10) Pub. No.: US 2010/0063360 A1
(43) Pub. Date: Mar. 11, 2010

(54) Side-arm Port Introducer

(75) Inventors: Douglas C. Harrington, San Jose, CA (US); Daniel P. Rogy, San Jose, CA (US)

Correspondence Address: CYTYC CORPORATION;Darry Pattinson, Sr. IP Paralegal, 250 CAMPUS DRIVE, MARLBOROUGH, MA, 01752, US

(73) Assignee: Adiana, Inc.

(21) Appl. No.: 11/564,244

(22) Filed: Nov. 28, 2006

(51) Int. Cl. A61B 1/015; A61B 1/012

(52) U.S. Cl. 600/159; 600/106

(57) ABSTRACT

A self-sealing introducer is disclosed that allows easy introduction of a catheter into a working channel of a surgical instrument while minimizing fluid leakage before, during and after catheter insertion.

### FIELD OF THE INVENTION

The inventions described below relate to the field of female sterilization.

### BACKGROUND OF THE INVENTION

In our prior U.S. Pat. No. 6,712,810, we disclosed a system for tubal occlusion which included a catheter with a small array of electrodes on its tip and a small foam plug lodged within the distal tip. The electrode array is used to wound the fallopian pathway, and the foam plug is dislodged from the catheter tip afterward. This method has proven to be highly effective and safe.

Proper placement of the catheter tip within the ovarian pathway is facilitated by inflating the uterus with a clear distention fluid and by viewing the placement through a hysteroscope, while the uterus is inflated. Placement is also facilitated by inserting the catheter into the uterus of the patient through the working channel of the hysteroscope. The working channel of the typical hysteroscope is entered through a side-arm access port, which is fitted with a ball valve to control passage through the access port. The ball valve may be closed when the working channel is not occupied to prevent leakage of the distention fluid that is used to inflate the uterus during the procedure. A sealing cap may be used to provide a rudimentary stasis over the distal end of the access port when the ball valve is open and a catheter occupies the working channel. Typical sealing caps merely comprise a small rubber cap with a hole in the middle of the cap, and do not serve as a stasis fitting as they allow significant outflow of distension fluid.

During the sterilization procedure, the physician can encounter several problems when placing the catheter in the working channel. Most importantly, if the surgeon forgets to open the ball valve before inserting the catheter, the catheter tip can be damaged when it is jammed into the closed ball valve. Though the typical sealing cap permits passage of robust catheter tips, delicate catheter tips may be damaged when forced through the hole of the sealing cap. Placement of the catheter in the working channel can be awkward for the physician, and the catheter tips may not be robust enough to be forced through the hole in the sealing cap. Also, distension fluid and bodily fluids can leak from the access port of the working channel before, during and after the catheter is inserted. The hole in the typical sealing cap limits outflow of distension fluid, but still allows an annoyingly energetic stream of distension fluid to exit toward the surgeon when feeding a catheter tip into the access port. What is needed is an introducer that can be easily handled by the physician that minimizes the leaking of fluids during a sterilization procedure and allows easy insertion of a fragile catheter tip assembly into a working channel.

### SUMMARY

The devices and methods described below provide for easy insertion of a working catheter tip into the side-arm access port of the typical hysteroscope while ensuring that the typical ball valve installed on the access port is open, thereby preventing the insertion of the catheter tip into the access port while the valve is closed. A side-arm introducer, comprising a tube adapted to fit in the lumen of the side-arm access port fixed to a stasis fitting adapted to fit over the proximal opening of the access port, is secured to the side-arm access port. A self-sealing membrane valve in the stasis fitting allows for easy introduction of a catheter while minimizing fluid leakage before, during and after catheter insertion. The proximal opening of the stasis fitting, into which the catheter tip is inserted, is funnel-shaped, with the self-sealing membrane valve disposed at the bottom of the funnel, and leads into a funnel shaped chamber which guides the catheter tip into the proximal opening of the tube. The stasis fitting disposed on the proximal end of the introducer tube is sized and dimensioned to fit most commercially available endoscopes or hysteroscopes, and the introducer tube is sized and fixed relative to the stasis fitting such that it intrudes in or through the lumen of the ball valve when the stasis fitting is secured to the proximal end of the access port.

### BRIEF DESCRIPTION OF THE DRAWINGS

FIG. 1 illustrates a catheter system developed for occlusion of the ovarian pathway of a female patient.

FIG. 2 is a close-up view of the distal tip of the catheter system of FIG. 1.

FIG. 3 illustrates a typical hysteroscope system used with the catheter of FIG. 1.

FIG. 4 illustrates a side-arm introducer.

FIG. 5 illustrates a sectional view of a side-arm introducer.

FIG. 6 illustrates a sectional view of the stasis fitting.

FIG. 7 illustrates a sectional view of a catheter inserted into a side-arm introducer disposed within an access port.

FIG. 8 illustrates the side-arm introducer in use.

FIG. 9 illustrates the side-arm introducer with the stasis fitting disposed about an access port.

### DETAILED DESCRIPTION OF THE INVENTIONS

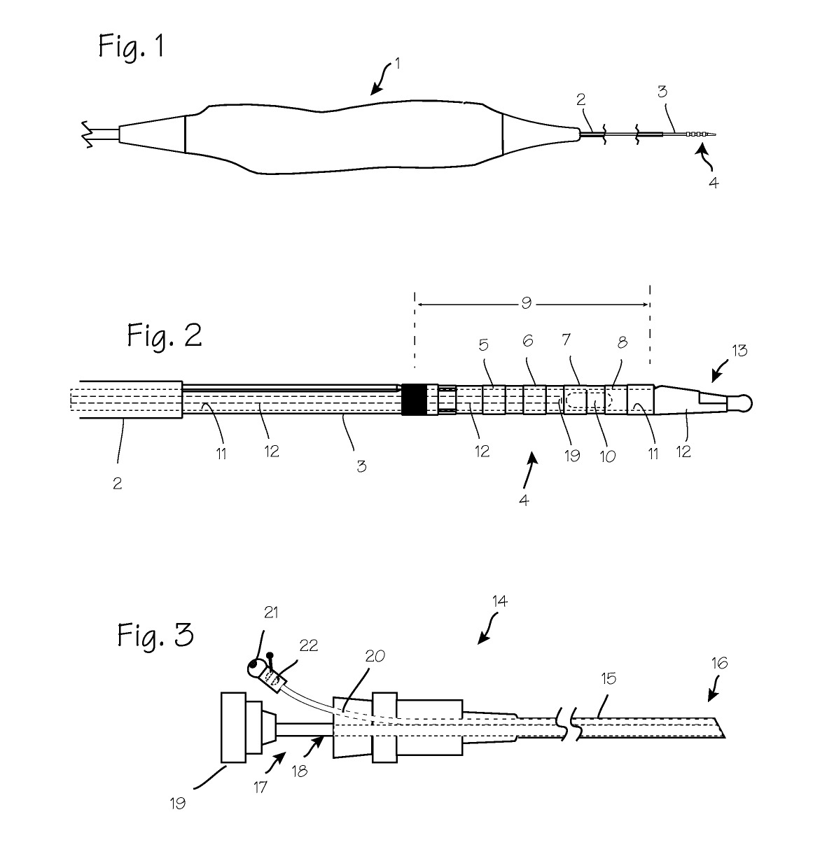

FIG. 1 illustrates a catheter system developed for occlusion of the ovarian pathway of a female patient. The catheter system 1 includes a catheter body 2 with a retractable sheath 3 slidably disposed within the catheter body. On the distal tip 4 of the sheath, four electrodes 5, 6, 7 and 8 aligned along the outer surface of the distal tip comprise a wounding segment 9. FIG. 2 is a close-up view of the distal tip of the catheter system of FIG. 1, illustrating the wounding segment 9 with the several electrodes disposed over the sheath 3, and the occlusive plug 10 stored within the wounding segment. The plug fits into the lumen 11 in the wounding segment of the catheter. A holding rod 12 is disposed within the catheter body 2, fixed longitudinally within the catheter body at any point proximal to the wounding segment which permits adequate pullback of the wounding segment to release the plug from slit 13 in the distal tip of the wounding segment. The distal tip and wounding segment are about 55 mil (about 1.4 mm) in outer diameter. For application of RF energy to the ovarian pathway as described in our U.S. Pat. No. 6,712,810, the wounding segment is about 6 to 8 mm long, and the electrodes are ring electrodes which are about 0.037 to 0.050 inches (0.9 to 1.3 mm) wide (measured along of the longitudinal axis of the catheter) and wrap around the catheter.

In use, the catheter is most conveniently inserted into the uterus transcervically through a hysteroscope, and the distal tip of the catheter is navigated into the fallopian tubes, until the wounding segment is stationed at the desired point along the ovarian pathway. The typical hysteroscope comprises a rigid endoscope with a working channel. FIG. 3 illustrates the construction of the typical hysteroscopic system or endoscopic system 14, which comprises a rigid tube 15 having a distal end 16 and a proximal end 17 with an hysteroscope 18 extending from an ocular lens 19 (or coupling for a camera, or electronic connector in the case of a CCD based scope) at the proximal end of the scope, through the rigid tube to an objective lens (or CCD in the case of a CCD based scope) at the distal end of the hysteroscope. A working lumen or channel 20, established by the gap between the endoscope and the rigid tube, extends from the proximal end of the scope to the distal end of the scope, terminating in an opening in the distal end of the scope. (Several other hysteroscopic arrangements are in use.) A side-arm access port 21 provides a conduit for inserting a tool, such as the catheter illustrated in FIG. 1, into the working channel so that the distal end may be advanced into the surgical site. The side-arm access port is typically accessed through a valve 22 because the hysteroscope is often used in procedures which require distension of the uterus with distension liquids or gasses at slight pressure, and the valve is used to prevent the distension fluid from escaping the uterus. The valve 22 is preferably a ball valve, the typical construction of which includes a ball with a smooth bored, straight lumen communication from the inlet (proximal, vis-à-vis tool insertion) and outlet (distal) sides of the valve. When tools are not occupying and occluding the working lumen, surgeons close the valve to prevent escape of distension fluid. Prior to inserting a working catheter, the surgeon opens the valve, tolerates some backflow of distension fluid, and inserts the working catheter. (The surgeon may cap the access port with a stasis fitting, with a small hole intended to accept a working catheter tip, but rather than stop distension fluid leakage this will cause squirting of distension fluid from the stasis fitting aperture.) If the surgeon forgets to open the valve before inserting the distal end of the working catheter, the distal end of the working catheter will be jammed into the ball of the ball valve. Delicate structures on catheters, such as the wounding segment shown in FIG. 1, may be damaged. The foam plug, for example, may be dislodged from its proper position, or the wounding segment may be crimped, such that the catheter should not be used. This is an expensive mistake, and typically requires replacement of the catheter. Other insertion catheter systems, such as those used for occlusive coils (for fallopian or vascular treatment), stents and other implants may be damaged during insertion.

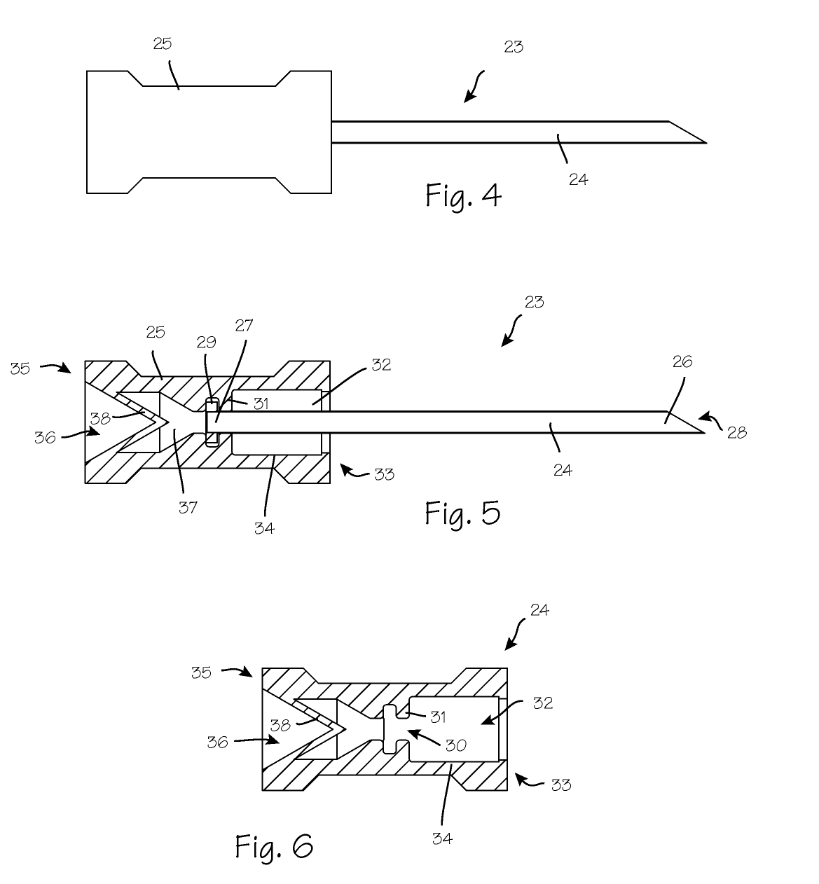

FIG. 4 and FIG. 5 illustrate a side-arm introducer 23 which, when installed in the side-arm port 21, prevents closure of the ball valve 22 and provides a substantial pressure seal to prevent leakage of distension fluid. As seen in FIG. 4, the side-arm introducer 23 comprises an introducer tube 24 engaging a self-sealing stasis fitting 25. FIG. 5 is a sectional view of the side-arm introducer 23. The introducer tube 24 is characterized by a distal end 26, a proximal end 27, and a lumen 28 extending through the introducer tube 24 defining a proximal opening and a distal opening. Distal end of introducer tube 24 may be shaped with an angular cut or formed into other suitable shapes. Flange 29 is fixed about proximal end of the introducer tube 24, and is trapped in the annular groove in the inside bore of the stasis fitting cylinder. The tube is approximately 1.6 inches (4 cm) in length, has an inner diameter of approximately 0.063 inches (1.6 mm) and outer diameter of approximately 0.068 inches (1.7 mm). With these dimensions, the introducer tube is sized and fixed relative to the stasis fitting such that it intrudes in or through the lumen of the ball valve when the stasis fitting is secured to the proximal end of the access port in a typical commercially available hysteroscopic system (the dimensions may be varied to accommodate other systems). The introducer tube 24 may be manufactured from a polymer such as polyproprylene, polyethylene, polyethylene terephthalate, polyimide and polybutylene terephthalate or other suitable materials. The size and length of the tube 24 may vary depending on the working catheter and hysteroscope with which it is used.

A sectional view of the stasis fitting 25 is illustrated in FIG. 6. The stasis fitting comprises a center bore 30 sized and dimensioned to frictionally engage the outer diameter of the introducer tube 24. A flange seat 31 is disposed within the stasis fitting 25 that is sized and dimensioned to accommodate the flange 29 when engaging the introducer tube 24. Thus, the flange 29 seats against the flange seat 31 when the introducer tube 24 is disposed within the stasis fitting 25. A counter bore 32 coaxial to the center bore is located on the distal section 33 of the stasis fitting. The counter bore 32 is sized and dimensioned to slip fit or frictionally engage an access port 21 or opening of a working channel of the hysteroscope (or other optical surgical device), or it may be formed with internal grooves to accommodate flanges or similar structures on the access port. The sidewall 34 of the counter bore 32 provides a seal between the outer surface of the access port of the working channel and the stasis fitting 25.

The proximal section 35 of the stasis fitting comprises a conical or funnel shaped recess 36. The conical recess is sized and dimensioned to accommodate, center and align a catheter with the introducer tube. A chamber 37 is disposed between the bottom wall 38 of the funnel 36 and the center bore 30. The distal section of the chamber 37 is also tapered, frusto-conical or funnel shaped to further assist in centering and aligning the catheter system with the introducer tube. When the introducer tube 24 is disposed within the stasis fitting 25, the introducer tube 24 is placed in fluid communication with the chamber 37. The bottom wall 38 of the funnel 36 on the stasis fitting's proximal section 35 is pierced upon insertion of the catheter tip, or it may be pierced or slit when manufactured, to create a membrane seal which is passable by the catheter but creates a fluid tight seal against the catheter that seals against fluid pressure. The wall 38 comprises self-sealing silicone membrane capable of accommodating the catheter by resiliently expanding and conforming around the catheter 2 when the catheter 2 is forced through the bottom wall 38 and resiliently closing to a fluid tight seal when the catheters are removed. Thus, the bottom wall 38 or fluid resistant seal allows for insertion of various instruments, catheters and elongate medical devices while functioning as a membrane seal that minimizes fluid leakage. The bottom wall 38 may also be provided with a slit (single or cross-cut) to facilitate insertion of the catheter or other medical instruments. In an alternative to the conical recess 36, the proximal section 35 of the stasis fitting may be provided with a check valve, duckbill valve, membrane valve, pinch valve or other such deformable self-sealing valve or fluid tight seal adapted to allow passage of the catheter through the stasis fitting 25 while limiting or preventing fluid flowing out of the working channel opening. Preferably, the introducer 23 allows no more than twenty or thirty grams of fluid to leak in a 10 minute period from a uterus inflated at about 3 psi of pressure (150-200 mm of mercury). The stasis fitting 25 is approximately 0.75 inches (about 20 mm) in length with a diameter of approximately 0.375 inches (about 10 mm), though the diameter and length of the stasis fitting 25 may vary depending on the application. The stasis fitting may be manufactured from silicone or other suitable materials including natural rubber, styrene block copolymers and thermoplastic polyurethanes.

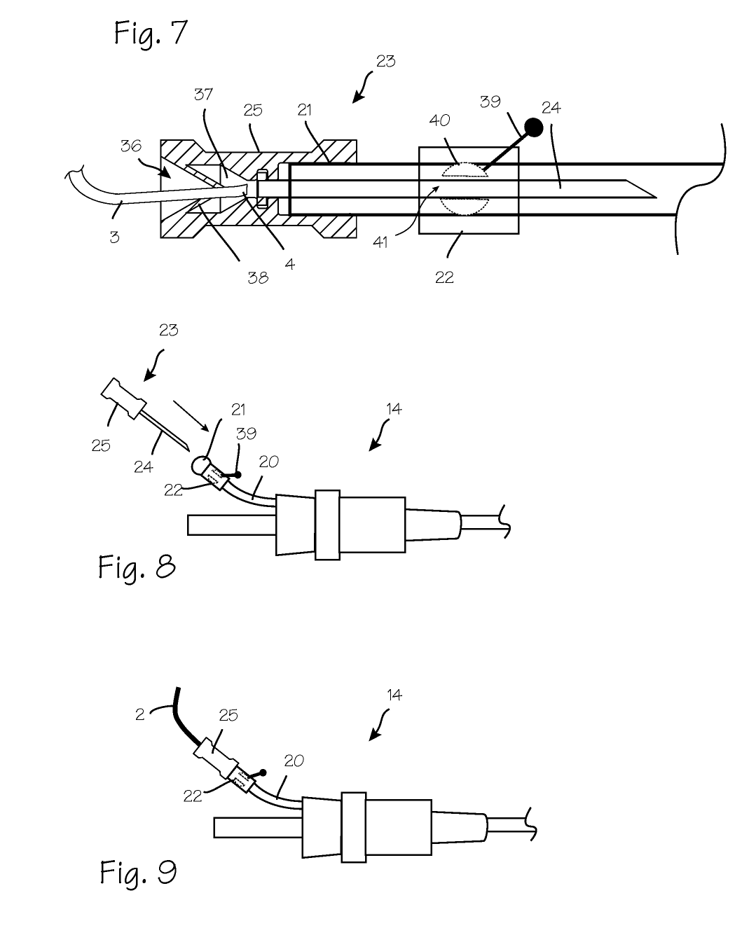

FIG. 7 illustrates a sectional view of a catheter 2 inserted into the access port 21 of a side-arm introducer 23. The ball valve 22 is operably disposed within the access port 21. The ball is a valve that opens by turning a handle 39 attached to a ball 40 inside the valve. The ball 40 has a hole 41 or smooth bored straight lumen through the middle so that when the hole is in line with both ends of the valve, flow will occur. When the valve is closed, the hole is perpendicular to the ends of the valve, and flow is blocked. Prior to insertion of the hysteroscopic system into the patient, the surgeon opens the ball valve and inserts the side-arm introducer so that stasis fitting fits over the access port and the tube extends through the ball valve. Alternatively, the surgeon may first insert the hysteroscopic system, open the valve and insert the side-arm introducer (some backflow of distension fluid may occur at this point) and then insert the working catheter 2. In either case, the access port is capped with the stasis fitting 25. Once the ball valve 22 is open and the side-arm introducer 23 is inserted, the ball valve cannot be shut inadvertently because introducer tube 24 interferes with the valve and blocks the valve open. The stasis fitting 25 is secured to the proximal end of access port 21, and holds the introducer tube in interfering relationship with the ball valve 22. The bottom wall 38 with a self-seal membrane valve at the apex of the funnel-shaped recess 36 in the proximal end of the stasis fitting has sealed around the catheter. The second funnel-shaped chamber 37 serves, as illustrated, to guide the distal tip 4 of the catheter into the proximal end of the introducer tube 24 in case the tip is curved or misaligned.

FIGS. 8 and 9 illustrates the use of the introducer with a hysteroscope. During a sterilization procedure, the physician inserts the introducer tube 24 into the access port 21 of a 0.070 inch working channel in a hysteroscope 14. If the valve 22 is closed, the introducer tube 24 will contact the ball 40, which will indicate to the physician that the valve needs to be opened. When the ball valve is open, the physician will completely insert the introducer tube 24 into the access port and at the same time releasably fit the stasis fitting 25 over the access port 21 by slip fitting the sidewall of the counter bore 32 of the stasis fitting over the outer surface of the access port. With the introducer installed in the side-arm access port 21, the ball valve 22 cannot be closed. The physician then inserts the hysteroscope to find and inspect the opening into the fallopian tubes (called the ostium). The physician inserts the transcervical catheter 2 through the bottom wall 38 of the conical recess 36 in the stasis fitting using only about 0.12 pounds (about 54 grams) of pressure, piercing the wall and forming a seal between the bottom wall 38 and the outer surface of the catheter 2. The seal prevents fluid from leaking out of the access port 21 during the surgical procedure. The transcervical catheter 2 is advanced through the introducer tube 24 and the working channel 20 until the catheter is seated in the ostium of the fallopian tube.

The physician then performs a sterilization procedure using the catheter. After sterilization has been confirmed (this may be done visually through the hysteroscope), the physician will remove the catheter. For complete sterilization, the physician will then repeat the procedure on the other side of the uterus. While identifying the contralateral ostia and preparing a second catheter for insertion, the insertion tube remains in place within the side-arm access part, with the ball valve in the open position and the stasis fitting substantially impeding any outflow of distension fluid through the working channel and side-arm.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A hysteroscopic system comprising:
a hysteroscope including a working channel and access port having a lumen communicating with the working channel, and a valve disposed in the access port;
an introducer comprising a tube adapted for insertion into the lumen of the access port and a stasis fitting fixed to the tube and adapted for attachment to the access port when the tube is inserted into the lumen;
wherein the tube is sized and dimensioned to extend through the valve when the stasis fitting is attached to the access port.

2. The hysteroscopic system of claim 4 wherein the stasis fitting comprises a center bore sized and dimensioned to frictionally engage the access port.

3. The hysteroscopic system of claim 4 wherein said stasis fitting has a funnel shaped recess at its proximal end, said funnel shaped recess terminating distally in a deformable self-sealing valve communicating with a funnel shaped chamber communicating with the proximal end of the tube.

4. A hysteroscopic system comprising:
a hysteroscope including a working channel and access port having a lumen communicating with the working channel, and a valve disposed in the access port; and
an introducer comprising a tube adapted for insertion into the lumen of the access port and a stasis fitting fixed to the tube and adapted for attachment to the access port when the tube is inserted into the lumen;
said stasis fitting having a funnel shaped recess at its proximal end, said funnel shaped recess terminating distally in a deformable self-sealing valve communicating with a funnel shaped chamber communicating with the proximal end of the tube.

5. The hysteroscopic system of claim 4 wherein the stasis fitting comprises a center bore sized and dimensioned to frictionally engage the access port.

6. The hysteroscopic system of claim 5 wherein the tube is sized and dimensioned to extend through the valve when the stasis fitting is attached to the access port.

7. An introducer for a working channel of an optical surgical device, where said optical surgical device comprises a working channel and an side-arm port communicating with said working channel, and a valve disposed in the side arm port or working cannel, said introducer comprising:
an insertion tube adapted for insertion into the working channel of the optical surgical device, said insertion tube having a distal end and a proximal end, and a lumen extending through the tube defining a proximal opening and a distal opening in the tube; and
a stasis fitting disposed about the proximal end of the tube;
wherein the tube is sized and dimensioned such that its distal end extends into interfering relationship with the valve when the stasis fitting is attached to the access port.

8. The introducer of claim 7, wherein the stasis fitting further comprises a funnel shaped recess at its proximal end, said funnel shaped recess terminating distally in a deformable self-sealing valve communicating with a funnel shaped chamber communicating with the proximal end of the tube.

9. The introducer of claim 8, wherein the self-sealing valve permits passage of a catheter through the self-sealing valve while substantially maintaining a fluid tight seal.
